(19) United States
(12) Patent Application Publication
(10) Pub. No.: US 2024/0050355 A1
(43) Pub. Date: Feb. 15, 2024

(54) TOPICAL SKIN TREATMENT COMPOSITION

(71) Applicant: Folashade Simpson, Woodland Hills, CA (US)

(72) Inventor: Folashade Simpson, Woodland Hills, CA (US)

(21) Appl. No.: 17/819,159

(22) Filed: Aug. 11, 2022

(51) Int. Cl. A61K 8/67; A61K 8/9783; A61K 8/34; A61Q 19/08

(52) U.S. Cl. CPC A61K 8/676; A61K 8/9783; A61K 8/342; A61Q 19/08; A61K 2300/00

(57) ABSTRACT

A composition for treating skin composed of water, L-ascorbic acid, aloe vera leaf juice, panthenol, flaxseed extract, glycerin, potassium sorbate, xanthan gum, tocopherol, sodium hydroxide, sodium benzoate, gluconolactone, and a fragrance.

### BACKGROUND OF THE INVENTION

The present invention relates to topical compositions, and more particularly, to a topical skin treatment composition.

Existing skin treatment compositions have many unnatural chemicals that may work in the short term, but negatively impact the skin in the long run. These solutions struggle to treat skin issues such as hyperpigmentation in the skin as well as aging, wrinkling skin.

As can be seen, there is a need for a topical skin treatment composition able to treat hyperpigmentation and aging, wrinkling skin without unnatural chemicals.

### SUMMARY OF THE INVENTION

In one aspect of the present invention, a composition for treating skin comprises water, L-ascorbic acid, aloe vera leaf juice, panthenol, flaxseed extract, glycerin, xanthan gum, tocopherol, sodium hydroxide, Panthenol, sodium benzoate, gluconolactone, and a fragrance.

The chemical composition of the serum of the present invention is scientifically proven to reduce dark-spots and promote youthful skin. The serum resolves the issue of hyperpigmentation in the skin as well as aging skin.

Advantageously, the skin care serum composition of the present invention provides an unprecedented advancement over previous skin care solutions. Other skin care serum compositions contain harmful chemicals which have not been tested on the skin for daily use and may negatively impact the skin with extended use.

The present invention will promote soft glowing skin, anti-aging skin, and the minimization of large pores while protecting against blemishes, scars, fine lines, and wrinkles.

These and other features, aspects and advantages of the present invention will become better understood with reference to the following drawings, description, and claims.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the present invention.

Broadly, an embodiment of the present invention provides topical hydration and/or moisturizing and treatment composition. The composition may erase dark spots, nutritiously hydrate skin, and protect against blemishes, hyperpigmentation, wrinkles, and scars.

The skin care composition of the present invention may be naturally derived from a combination of naturally occurring acids, vitamins, and Alpha-linolenic acid—a type of Omega-3 fatty acids. The skin care serum composition of the present invention includes no known harmful ingredients and may therefore avoid negative impact on the skin of the user.

The skin care composition of the present invention may comprise a composition having about 97% natural and plant derived components. These components, advantageously, pose minimal, if any, risk to human health. The skin care serum composition of the present invention may be safely used for skin care and cosmetic use.

In some embodiments, the skin care serum of the present invention may comprise of one or more of the following components or elements and combinations thereof: Purified Water (Aqua), Glycerin, Vitamin E (Mixed Tocopherols), Linum Usitatissimum Extract (Flaxseed Extract), Aloe Barbadensis Leaf Juice, Panthenol, L-Ascorbic Acid, Xanthan Gum, Sodium Hydroxide, Sodium Benzoate, Gluconolactone, and Fragrance.

The components may be quantified as a percentage which, when homogenously mixed, makes the total solution 100%. The components work together providing Omgea-3 fatty acids and key vitamins that improve the skins moisture, elasticity, and complexion.

Fragrances are not particular to the present invention and any fragrance may be added for olfactory purposes.

In some embodiments, the skin care serum composition of the present invention may be made by a process including one or more of the following steps and combinations thereof. In some embodiments, the steps may be followed in a different order than listed. In some embodiments varying percentages of ingredients may be used.

The Flaxseed Extract (Linum Usitatissimum Extract) is formed by boiling the Flaxseed to create a gel like substance. Alternatively, the Flaxseed Extract may be created by soaking the Flaxseeds for up to a few hours to a few days. This too will create the gel like substance. Either Golden Flaxseed and Brown Flaxseed may be used or a combination thereof. Other types of I-ascorbic acid and/or Vitamin C may be used including but not limited to the following: ascorbyl-6-palmitate, Ascorbic Acid, Sodium Ascorbyl Phosphate, Magnesium Ascorbyl Phosphate, Sodium Ascorbate, Calcium Ascorbate, and Ascorbyl Palmitate. All species and varieties of medicinal Aloe Vera may be used including but not limited to the following: Aloe Barbadensis Miller, Aloe Arborescens, Aloe Saponaria, and Aloe Ferox.

The present invention may be composed of the following ingredients expressed in percentages by weight:

Water—between about 10% and 80%
L-Ascorbic Acid—between about 0.05% to 20%
Aloe Vera Leaf Juice—between about 0.05% to 87%
Vitamin B5 (Panthenol)—between about 0.05% to 20%
Flaxseed—between about 0.05% to 90%
Glycerin—between about 0.05%-20%
Potassium Sorbate—between about 0.05% to 2.0%
Xanthan Gum—between about 0.05% to 2.0%
Vitamin E, Mixed Tocopherols—between about 0.05% to 20%
Sodium Hydroxide—between about 0.05% to 2.0%
Sodium Benzoate—between about 0.05% to 2.0%
Gluconolactone—between about 0.05% to 2.0%
Fragrance—between about 0.05% to 2.0%

In some embodiments, the skin care composition of the present invention may be made by a process including one or more of the following method steps and combinations thereof as described below. In some embodiments, the steps may be followed in a different order than listed.

TABLE 1
Compositions and Phases for Formulation of the Present Invention

| | | INCI (International | | |
|---|---|---|---|---|
| | | Nomenclature of | | |
| Phase | Tradename | Cosmetic Ingredients) | Function | Weight % |
| A | Water | Water | Solvent | 65.50% |
| A | Vitamin B5 | dl-Panthenol | Moisturizing, | 2.00% |
| | | | Soothing | |
| A | L-Ascorbic Acid | L-Ascorbic Acid | Lightening, | 10.00% |
| | | | Collagen | |
| | | | Boosting | |
| A | Aloe Vera Leaf | Aloe Barbadensis | Moisturizing, | 5.00% |
| | Juice | (Aloe Vera) Leaf Juice | Soothing | |
| A | Flaxseed Extract | Linum Usitatissimum | Anti-ageing, | 10.00% |
| | | (Flaxseed) Extract | Anti-wrinkle | |
| B | Glycerin | Glycerin | Humectant | 3.00% |
| B | Xanthan Gum | Xanthan Gum | Rheology | 0.40% |
| | | | Modifier | |
| C | Vitamin E, Mixed | Tocopherol | Antioxidant | 0.10% |
| | Tocopherols T50 | | | |
| C | Preservative | Geoguard Ultra 35 | Preservative | 2.00% |
| | | (Sodium Benzoate and/or | | |
| | | Gluconolactone) | | |
| D | Caustic Soda | Sodium Hydroxide | pH adjuster | 2.0% |

1. Combine ingredients of phase A under low shear until homogenously mixed forming a first solution;
2. Combine ingredients of phase B under low shear until a slurry forms. Add to phase A and stir under high shear until homogenous;
3. Combine materials of phase C to form a second solution. Add slowly to phase first solution of the A/B under low shear. Stir until homogenous; and
4. Check/adjust pH to 4-4.25 by adding material of phase D as needed. Give the product a final stir the next day before packing off.

The skin care composition of the present invention may be used for skin care and cosmetic use. In some embodiments, the skin care composition of the present invention may be used in the following manner. The skin care composition of the present invention may be used as a facial skin treatment serum twice daily, once in the morning, and once in the evening. In some embodiments the skin care composition of the present invention may be used on skin at areas of the body other than the face. The present invention may be used as a facial skin serum or a body skin serum twice daily, once in the day, and once at night.

For clarity, only those aspects germane to the invention are described, and product details well known in the art are omitted. In addition, many embodiments of the present invention have application to a wide range of industries. To the extent the present application discloses a system, the method implemented by that system is within the scope of the present invention. Further, to the extent the present application discloses a method, a system of apparatuses configured to implement the method are within the scope of the present invention.

It should be understood, of course, that the foregoing relates to exemplary embodiments of the invention and that modifications may be made without departing from the spirit and scope of the present invention.

What is claimed is:

1. A composition for treating skin comprising the following ingredients by weight percentage:
water: 10%-80%;
L-ascorbic acid: 0.05% to 20%;
aloe vera leaf juice: 0.05% to 87%;
panthenol: 0.05% to 20%;
flaxseed extract: 0.05% to 90%;
glycerin: 0.05%-20%;
potassium sorbate: 0.05% to 2.0%;
xanthan gum: 0.05% to 2.0%;
tocopherol: 0.05% to 20%;
sodium hydroxide: 0.05% to 2.0%;
sodium benzoate: 0.05% to 2.0%;
gluconolactone: 0.05% to 2.0%; and
a fragrance: 0.05% to 2.0%.

2. The composition of claim 1, wherein the weight percentages are:
water: 65.50%;
L-ascorbic acid: 10.00%;
aloe vera leaf juice: 5.00%;
panthenol: 2.00%;
flaxseed extract: 10.00%;
glycerin: 3.00%;
xanthan gum: 0.40%;
tocopherol: 0.10%;
sodium hydroxide: 2.0%; and
sodium benzoate: 2.0%.

3. The composition of claim 2, wherein the composition is formed by the method steps of:
combining water, panthenol, L-ascorbic acid, aloe vera leaf juice, and flaxseed extract under a low shear until a first solution is formed wherein the first solution is mixed until homogenous;
combining glycerin and xanthan gum at a low shear until a slurry forms;
adding the slurry to the first solution and mixing at a high shear until the slurry is homogenously mixed into the first solution;
combining tocopherol and sodium benzoate to form a second solution; and
adding the second solution to first solution and mixing at a low sheer until the second solution is homogenously mixed into the first solution.

4. The composition of claim 3, further comprising the method step of:
adding sodium hydroxide into the first solution until a pH range of 4-4.25 is achieved.
